(19) United States
(12) Patent Application Publication
(10) Pub. No.: US 2020/0094818 A1
(43) Pub. Date: Mar. 26, 2020

(54) VEHICLE CONTROL SYSTEM, VEHICLE, AND VEHICLE CONTROL METHOD

(71) Applicant: BOE TECHNOLOGY GROUP CO., LTD., Beijing (CN)

(72) Inventors: Xinguo LI, Beijing (CN); Wenbo LI, Beijing (CN)

(21) Appl. No.: 16/618,842

(22) PCT Filed: Mar. 27, 2019
(86) PCT No.: PCT/CN2019/079848

§ 371 Date: Dec. 3, 2019

(30) Foreign Application Priority Data
Mar. 27, 2018 (CN) 201810258784.6

(51) Int. Cl. B60W 30/08; B60W 10/04; B60W 10/18; B60W 10/30; G07C 5/08; A61B 5/16; A61B 5/0205; A61B 5/0476; A61B 5/18; A61B 5/00

(52) U.S. Cl. CPC B60W 30/08; B60R 21/01512; B60W 10/18; B60W 10/30; G07C 5/08; A61B 5/163; A61B 5/0205; A61B 5/0476; A61B 5/18; A61B 5/4809; A61B 5/165; B60W 2030/082; B60W 2720/106; B60W 2710/18; B60W 2540/26; B60W 10/04

(57) ABSTRACT

A vehicle control system, a vehicle and a vehicle control method are provided, and the vehicle control system includes: an acquiring device configured to acquire body state information of a driver; a processor, which includes an input terminal connected with the acquiring device, and is configured to judge whether the body state information is body abnormality information, and to output an execution signal if the body state information is the body abnormality information; and an execution device, which includes an input terminal connected with an output terminal of the processor, and is configured to receive the execution signal, and to execute a corresponding security action according to the execution signal.

This application claims the benefit of Chinese patent application No. 201810258784.6 filed on Mar. 27, 2018, which is hereby entirely incorporated by reference as a part of the present application.

### TECHNICAL FIELD

Embodiments of the present disclosure relate to a vehicle control system, a vehicle and a vehicle control method.

### BACKGROUND

With rapid developments of vehicle technologies, vehicles have become a very important means of transportation in people's lives, making people's travel and trip more convenient. With the popularity of the vehicles, vehicle driving security has also attracted more and more attentions from people. However, a driver often loses control of the vehicle while driving due to subjective reasons, such as lack of concentration, sleep during driving, etc., which causes a vehicle accident and seriously threatens the driver's driving security.

### SUMMARY

The embodiments of the present disclosure provide a vehicle control system, including: an acquiring device configured to acquire body state information of a driver; a processor, which includes an input terminal connected with the acquiring device, and is configured to judge whether the body state information is body abnormality information and to output an execution signal in a case where the body state information is the body abnormality information; and an execution device, which includes an input terminal connected with an output terminal of the processor to receive the execution signal and to execute a corresponding security action according to the execution signal.

For example, the vehicle control system further includes a vehicle detector configured to detect vehicle state information of a vehicle; the input terminal of the processor is further connected with the vehicle detector, and the processor is further configured to judge whether the vehicle state information is vehicle abnormality information and to output the execution signal in a case where the vehicle state information is the vehicle abnormality information.

For example, the acquiring device includes a detection device which is configured to be in the vehicle and to be corresponding to a position of the driver. The detection device is provided with at least one selected from the group consisting of a sound detector, a camera and an eyeball detector; the sound detector is configured to detect sound information which is in the vehicle and is used as the body state information, the camera is configured to acquire facial image information which is used as the body state information, and the eyeball detector is configured to acquire eyeball information of the driver, which is used as the body state information.

For example, the acquiring device further includes a brain wave detector which is configured to be worn on a head of the driver, an output terminal of the brain wave detector is connected with the processor, and the brain wave detector is configured to detect brain wave information of the driver, which is used as the body state information.

For example, the vehicle detector is configured to be provided at the vehicle and includes at least one selected from the group consisting of a vibration detector, a collision detector, a tilt detector and a water pressure detector. The collision detector is configured to detect collision information of the vehicle, which is used as the vehicle state information; the vibration detector is configured to detect vibration information of the vehicle, which is used as the vehicle state information; the tilt detector is configured to detect tilt information of the vehicle, which is used as the vehicle state information; and the water pressure detector is configured to detect vehicle body water pressure information of the vehicle, which is used as the vehicle state information.

For example, the execution device includes at least one selected from the group consisting of a brake vehicle control system, a gasbag opener vehicle control system, a glass breaker vehicle control system and an alarm vehicle control system.

The embodiments of the present disclosure further provide a vehicle including the vehicle control system.

The embodiments of the present disclosure further provide a vehicle control method, including: acquiring body state information of a driver of a vehicle; judging whether the body state information is abnormal driving information, and controlling a vehicle to execute a corresponding security action in a case where the body state information is the abnormal driving information.

For example, the vehicle control method further includes: acquiring vehicle state information of the vehicle; judging whether the vehicle state information is abnormal vehicle information; judging whether the body state information is the abnormal driving information; and controlling the vehicle to execute the corresponding security action in a case where the vehicle state information is the abnormal vehicle information and/or the body state information is the abnormal driving information.

For example, the body state information includes at least one selected from the group consisting of sound information, facial expression information, eyeball information, heart rate information and brain wave information; the vehicle state information includes at least one selected from the group consisting of collision information, vibration information, tilt information and water pressure information; the security action includes at least one selected from the group consisting of a braking action, a deceleration action, a gasbag opening action and a glass breaking action.

### BRIEF DESCRIPTION OF THE DRAWINGS

In order to clearly illustrate the technical solution of the embodiments of the disclosure, the drawings of the embodiments will be briefly described in the following; it is obvious that the described drawings are only related to some embodiments of the disclosure and thus are not limitative of the disclosure.

FIG. 1 is a structural block diagram of a vehicle control system according to an embodiment of the present disclosure;

FIG. 2 is a structural block diagram of the vehicle control system according to another embodiment of the present disclosure;

FIG. 3 is a schematic structural diagram of an acquiring device according to an embodiment of the present disclosure;

FIG. 4 is a schematic structural diagram of a vehicle detector according to an embodiment of the present disclosure;

FIG. 5 is a schematic structural diagram of an execution device according to an embodiment of the present disclosure;

FIG. 6 is a flowchart of a vehicle control method according to an embodiment of the present disclosure; and

FIG. 7 is a flowchart of the vehicle control method according to another embodiment of the present disclosure.

### DETAILED DESCRIPTION

In order to make objects, technical details and advantages of the embodiments of the disclosure apparent, the technical solutions of the embodiments will be described in a clearly and fully understandable way in connection with the drawings related to the embodiments of the disclosure. Apparently, the described embodiments are just a part but not all of the embodiments of the disclosure. Based on the described embodiments herein, those skilled in the art can obtain other embodiment(s), without any inventive work, which should be within the scope of the disclosure.

Unless otherwise defined, all the technical and scientific terms used herein have the same meanings as commonly understood by one of ordinary skill in the art to which the present disclosure belongs. The terms “first,” “second,” etc., which are used in the description and the claims of the present application for disclosure, are not intended to indicate any sequence, amount or importance, but distinguish various components. Also, the terms such as “a,” “an,” etc., are not intended to limit the amount, but indicate the existence of at least one. The terms “comprise,” “comprising,” “include,” “including,” etc., are intended to specify that the elements or the objects stated before these terms encompass the elements or the objects and equivalents thereof listed after these terms, but do not preclude the other elements or objects. The phrases “connect”, “connected”, etc., are not intended to define a physical connection or mechanical connection, but may include an electrical connection, directly or indirectly. “On,” “under,” “right,” “left” and the like are only used to indicate relative position relationship, and when the position of the object which is described is changed, the relative position relationship may be changed accordingly.

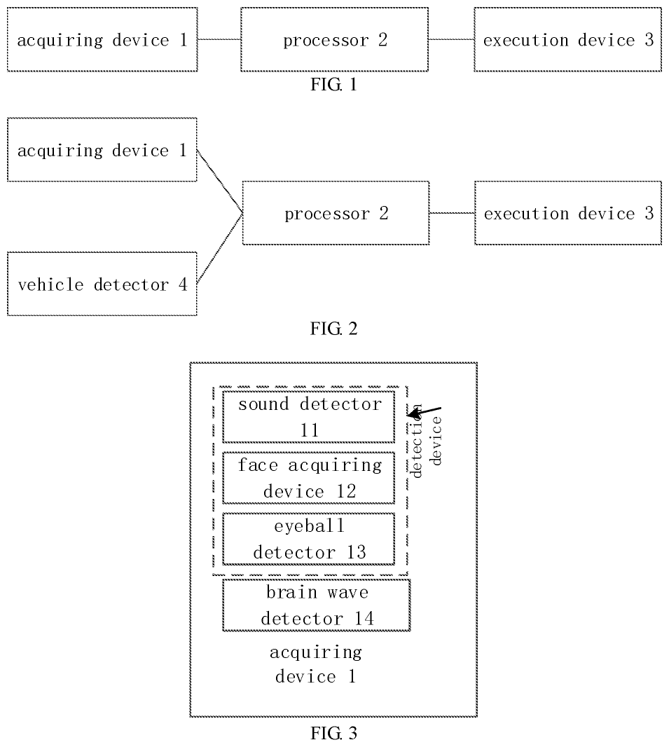

As shown in FIG. 1, the embodiments of the present disclosure provide a vehicle control system including: an acquiring device 1 configured to acquire body state information of a driver; a processor 2, which includes an input terminal connected with the acquiring device 1, and is configured to judge whether the body state information is body abnormality information and to output an execution signal in a case where the body state information is the body abnormality information; and an execution device 3, which includes an input terminal connected with an output terminal of the processor 2, and is configured to receive an execution signal and to execute a corresponding security action according to the execution signal.

The acquiring device 1 is configured to acquire the body state information of the driver. The body state information may include various types of information, such as: brain wave information of a human body, eye closing information of the human body, sound information of the human body, facial expression information of the human body, heartbeat information of the human body and the like. In order to obtain the above-described body state information, the acquiring device 1 may include detection devices corresponding to the above-described body state information, such as: a brain wave sensor, a camera, a heart rate sensor, a sound sensor and the like, which are not limited here. It should be noted that the acquiring device 1 may detect the body state information of the driver in real time and transmit the detected body state information to the processor 2, so that a judgment can be performed in real time and the security of the driver can be improved.

The processor 2 is configured to receive the body state information of the driver detected by the acquiring device 1 and to judge the body state information. In a case where the detected body state information is the body abnormality information, it is indicated that the present body state of the driver is not suitable for keeping driving, or that the driver loses ability or consciousness for controlling the vehicle, which is very dangerous. The judgment criteria of the body abnormality information may be varied depending on the different types of the detected body state information. For example, in a situation where it is detected that the eye closing time of the driver exceeds a preset time, for example, the preset time is 5 seconds, it is judged that the driver is in a fatigue driving state, which is very dangerous and is prone to traffic accidents, and emergency stop may be performed by the execution device 3. For another example, in a situation where the sound information of the driver is detected to be screaming sound information, it is judged that a danger occurs during the driving, and that the driver may lose control of the vehicle, which is very dangerous, and emergency control may be performed by the execution device 3. For still another example, in a situation where it is detected that the heart rate information of the driver exceeds a preset value, it is judged that the driver is very nervous or excited at this time and may lose the ability of controlling the vehicle, and emergency braking may be performed by the execution device 3. According to the above examples, the processor 2 is configured to judge whether the driver is in an abnormal driving state based on the body state information of the driver, and the judgment criteria can be specifically set according to the type of the body state information; once the present body state information of the driver is judged to be the body abnormality information, the processor 2 outputs the execution signal to control the execution device 3 to act, so as to ensure the security of the driver. It should be noted that the processor 2 may be a vehicle controller in the vehicle or a controller that is additionally added to the existing vehicle, and the specific structural form of the processor is not limited here.

The above-mentioned execution device 3 is configured to execute the corresponding security action according to the acquired execution signal, and the execution device 3 may be a plurality of structural components in the vehicle, such as a brake, a gasbag opener, an alarm and so on. The execution device 3 may automatically control the vehicle to issue an alarm to remind the driver, so as to ensure the driving security of the driver. In a situation where the above-mentioned execution device 3 receives the execution signal from the processor 2, the execution device 3 performs the corresponding security action such as braking, opening the gasbag, alarming or the like, which are not specifically limited heren.

Embodiments of the present disclosure provide the vehicle control system for driving security of the driver. In conventional technologies, the driver loses control of the vehicle often due to subjective reasons of the driver in driving while driving, causing a serious vehicle accident and seriously affecting the security of the society. Compared with the conventional technologies, the vehicle control system provided by the present application includes the acquiring device 1, the processor and the execution device; when the driver drives the vehicle, the acquiring device 1 may acquire the body state information of the driver, and send the body state information to the processor; the processor judges whether the body state information is the body abnormality information, and in a case where the body state information is the body abnormality information, the processor sends the execution signal to the execution device, and the execution device may execute the corresponding security action according to the execution signal, so that in a situation where the driver experiences a subjective uncontrollable phenomenon during driving, such as falling asleep, screaming or the like, the vehicle control system automatically controls the vehicle through the execution device, to ensure the security of the driver.

Besides detecting and judging the body state information of the driver, the above vehicle control system may also detect and determine a state information of the vehicle itself. As shown in FIG. 2, for example, the vehicle control system further includes a vehicle detector 4, and the vehicle detector 4 is configured to detect vehicle state information of the vehicle; the input terminal of the processor 2 is connected with the vehicle detector 4, and the processor 2 is configured to judge whether the vehicle state information is vehicle abnormality information and to output the execution signal in a case where the vehicle state information is the vehicle abnormality information. In this embodiment, the vehicle detector 4 is configured to detect the vehicle state information of the vehicle, the vehicle state information may include vibration information, collision information, tilt information and the like of the vehicle, and the vehicle state information may reflect the form and state that the vehicle is in. In order to acquire the above-described vehicle state information, the above-described vehicle detector 4 may be a vibration sensor, a collision sensor, a tilt sensor or the like. The above-described vehicle state information may be transmitted to the processor 2 by the vehicle detector, and the processor 2 may judge whether the vehicle state information is the vehicle abnormality information according to the vehicle state information. The vehicle abnormality information includes, for example, too large vibration amplitude, occurrence of collision, too large tilt amplitude, entrance of water into the vehicle or the like. If the vehicle state information is the vehicle abnormality information, the execution signal is sent to the execution device 3, so as to control the execution device 3 to execute the security action, and the security action includes, for example, a braking action, a security gasbag opening action, a glass breaking action or the like, which are not limited here, so that the driver's personal security can be protected.

The above-described acquiring device 1 may be of various types, as shown in FIG. 3. For example, the acquiring device 1 includes a detection device configured to be disposed in the vehicle and corresponding to a position of the driver. In some examples, the detection device includes at least one selected from the group consisting of a sound detector 11, a face acquiring device 12 and an eyeball detector 13; the sound detector 11 is configured to detect sound information which is in the vehicle for the body state information; the face acquiring device 12 is configured to capture facial image information of the driver for the body state information; the eyeball detector 13 is configured to detect eyeball information of the driver for the body state information. In this embodiment, the detection device may be disposed in the interior of the vehicle and be opposite to a front of a position of the driver, so that the detection device can detect the body state information of the driver more conveniently, so as to ensure an accuracy of the detected data. For example, any one or more of the sound detector 11, the face collection 12 and the eyeball detector 13 may be integrated in the detection device.

For example, the sound detector 11 includes a device such as a microphone or the like to acquire the sound information of the driver. During the driving process of the vehicle, in a situation where the vehicle is in an emergency, the driver may scream and lose control of the vehicle; by the processor 2, it may be judged whether the sound information of the driver acquired by the sound detector 11 is abnormal state information, such as a screaming sound or the like; and the processor 2 may control the execution device 3 (for example, a brake) to execute a security action (for example, braking action) to reduce or avoid injuries or accidents.

For example, the face acquiring device 12 may include an image acquiring device, such as a camera, for acquiring the facial image information of a human face; by the processor 2, whether the state of the driver is normal may be analyzed based on the facial image information. For example, in a situation where an emergency occurs, the driver may cover the face with hand, or the facial expression of the driver may be fearsome. The processor 2 may compare the facial image information with a data base of abnormal facial image information; if the facial image information is found to be the abnormal state information, the processor 2 may control the execution device 3 to execute the security action, such as braking or the like, to ensure the security of the driver.

For example, the eyeball detector 13 is configured to detect eyeball information such as a closing state of the eyeball, a gazing direction of the eyeball and the like; and by the processor 2, whether the driving state of the driver is normal may be analyzed based on the eyeball information. For example, in a situation where the eye closing time indicated by the eyeball information is too long, it may be judged that the driver falls asleep; in a situation where the gazing direction of the eyeball indicated by the eyeball information is shifted for a long time, it may be judged that the attention of the driver is not concentrated on driving, so that the processor 2 can judge whether the eyeball information is the abnormal state information based on the condition of the eyeball information. In a situation where the eyeball information is determined as the abnormal state information, the execution device 3 may be controlled to execute the security action such as braking, alarming or the like, to ensure the security of the driver. The structural types of the eyeball detector 13 may be various. For example, the eyeball detector 13 may include an infrared light emitter and an eyeball position sensor, the infrared light emitter is configured to emit infrared light, and the eyeball position sensor includes an infrared light detector configured to receive the infrared light reflected by an eye (such as an eyeball and a cornea), so as to capture actions such as a position movement of the eyeball, eye blinking and eye closing, which are not specifically limited in the embodiments of the present disclosure.

The above-described acquiring device 1 may further include a brain wave detector 14 as shown in FIG. 3, in addition to the detection device. For example, the acquiring device 1 includes a brain wave detector 14 configured to be worn on the head of the driver, an output terminal of the brain wave detector 14 is connected with the processor 2, and the brain wave detector 14 is configured to detect brain wave information of the driver for the body state information. In this embodiment, the brain wave detector 14 may be worn on the head of the driver. For example, the brain wave detector 14 may be implemented as a headgear structure, an eyewear structure, or the like, and the specific structure of the brain wave detector 14 is not limited herein. For example, the brain wave detector 14 includes a detection electrode configured to contact the head to receive and transmit an electrical signal generated by a brain wave. For example, the detection electrode is a metal electrode. For example, the brain wave detector 14 and the processor 2 may be in a wired connection or in a wireless connection. For example, the wireless connection may be a Bluetooth connection, a wireless fidelity connection or the like. The connection manner between the brain wave detector 14 and the processor 2 is not limited. The brain wave detector 14 is configured to detect the brain wave information of the driver and to transmit the brain wave information as the body state information to the processor 2, and the processor 2 may determine whether the driver is in the abnormal driving state by analyzing the brain wave information (e.g. a brain wave disorder) of the driver. If the brain wave information is judged to be the body abnormality information, the corresponding execution device 3 may be controlled to execute the security action such as braking, alarming or the like, to ensure the security of the driver.

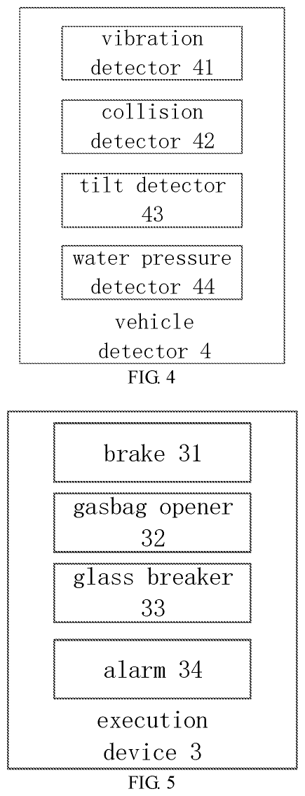

The above-described vehicle detector 4 may be of various types in structure. For example, the vehicle detector 4 is provided at the vehicle. As shown in FIG. 4, the vehicle detector 4 includes at least one selected from the group consisting of a vibration detector 41, a collision detector 42, a tilt detector 43 and a water pressure detector 44; the collision detector 42 is configured to detect collision information of the vehicle as the vehicle state information; the vibration detector 41 is configured to detect vibration information of the vehicle as the vehicle state information; the tilt detector 43 is configured to detect tilt information of the vehicle as the vehicle state information; the water pressure detector 44 is configured to detect a water pressure information of the vehicle as the vehicle state information. In this embodiment, the vibration detector 41 is used to detect the vibration information of the vehicle, and the processor 2 may determine the state of the vehicle body according to the vibration condition of the vehicle. For example, if the vehicle vibrates greatly, it may be judged that the vehicle travels dangerously, that it is not suitable for the vehicle to drive too fast, and even that the vehicle needs to be braked; in this case, the processor 2 may control the execution device 3 to correspondingly execute the security actions such as braking or deceleration or the like, to ensure driving security. The collision detector is configured to detect the collision information of the vehicle; in a situation where the processor 2 acquires the collision information, it may be judged that collision occurs to the vehicle, and in order to prevent the driver from being unable to control the vehicle due to being nervous or unconscious, the processor 2 may automatically control the execution device 3 to execute the security action, such as braking, opening the gasbag or the like, to ensure the security of the driver, and to prevent the accident from expanding. The tilt detector is configured to detect the tilt state of the vehicle; if a tilt angle is large, the processor 2 may determine that the vehicle may flip over; in this case, the processor 2 may control the corresponding gasbag to open, so as to protect the security of the driver. The water pressure detector 44 is configured to detect the water pressure information outside the vehicle; if the water pressure appears at the glass of the vehicle, it may be judged that the vehicle may fall into water; and in this case, the processor 2 may control the corresponding execution device 3, which is, for example, a glass breaker to break the glass of the vehicle, so that the driver can escape the vehicle through a window to ensure the security of the driver.

For example, the vibration detector 41 includes a vibration sensor, the collision detector 42 includes an acceleration sensor, the tilt detector 43 includes a tilt angle sensor and a GPS positioner, and the water pressure detector 44 includes a pressure sensor. The embodiments of the present disclosure are not limited thereto.

The execution device is shown in FIG. 5, for example, and the execution device 3 includes at least one selected from the group consisting of a brake 31, a gasbag opener 32, a glass breaker 33 and an alarm 34. In this embodiment, the processor 2 may control the brake 31 to perform the security action such as deceleration, braking or the like; the gasbag opener 32 may execute the security action such as opening the gasbag or the like; the glass breaker 33 may realize the security action such as breaking the glass of the vehicle or the like; the alarm 34 may execute the security action such as danger warning or the like to remind the driver of a bad state, so that the driver can make corrections in time to avoid traffic accidents. The above multiple execution devices 3 may be executed simultaneously in multiple combinations, or may be executed separately or sequentially, and the execution manner of the execution devices may be set according to the specific judgment of the processor, and is not specifically limited here.

For example, the processor 2 may be a central processing unit (CPU), a digital signal processor (DSP), or other forms of processor with data processing capability and/or program execution capability, such as a field programmable gate array (FPGA) or the like. For example, the central processing unit (CPU) may be an X86 or ARM architecture or the like. The processor may be a general purpose processor or a special purpose processor, and may control the execution device 3 to execute a desired function. For example, the gasbag opener includes a sensor, a gas generator, a gasbag and a controller. The sensor is configured to sense a degree of collision of the vehicle and send a signal to the controller. The controller is configured to process the signal and send a signal to trigger the gas generator in a situation where it is determined that it is necessary to open the gasbag. The gas generator is configured to quickly ignite and generate a large amount of gas to inflate the gasbag, so as to open the gasbag.

For example, the glass breaker may be a tool that can be used to break the glass of the window of the vehicle, such as a glass puncher, a glass cutter, a security hammer, or the like.

For example, the vehicle control system further includes a memory for storing computer-readable instructions (e.g., one or more computer program modules) non-transiently, and the computer-readable instructions may be run by the processor to control the execution device 3 to execute a desired action. The memory and processor may be interconnected via a bus system and/or other form of connection mechanism (not shown).

Embodiments of the present disclosure also provide a vehicle including the vehicle control system described above.

Embodiments of the present disclosure provide a vehicle for protecting the driving security of the driver. In conventional technologies, the driver often loses control of the vehicle while driving due to subjective reasons, causing a serious vehicle accident and seriously affecting the security of the society. The vehicle provided by the embodiments of the present disclosure includes an acquiring device, a processor and an execution device. during the driving process that the driver drives the vehicle, the acquiring device may acquire a body state information of the driver, and send the body state information to the processor; the processor judges whether the body state information is body abnormality information, and sends an execution signal to the execution device in a case where the body state information is the body abnormality information; the execution device may execute a corresponding security action according to the execution signal, so that in a situation where the driver experiences a subjective uncontrollable phenomenon during driving, such as falling asleep, screaming or the like, the vehicle control system automatically can control the vehicle through the execution device, to ensure the security of the driver.

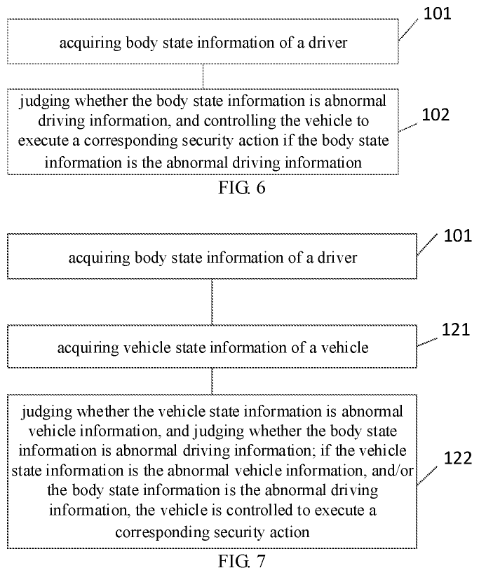

As shown in FIG. 6, the embodiments of the present disclosure further provide a vehicle control method, which at least includes: step 101, acquiring a body state information of the driver, in which step, the body state information may reflect a driving state of the driver, and the body state information may include a plurality of types of information, for example, brain wave information of the human body, eye closing information of the human body, sound information of the human body, facial expression information of the human body, heartbeat information of the human body, and the like; and step 102, judging whether the body state information is abnormal driving information, and controlling the vehicle to execute a corresponding security action if the body state information is the abnormal driving information.

The body state information can reflect the body condition of the driver, so as to know whether the driver still has an ability to control the vehicle. If the driver may be unable to control the vehicle, it can be regarded as the abnormal driving state. The abnormal driving state may have a variety of types, for example: the driver's brain wave is disordered, the driver's heart rate is too fast, the driver closes the eye for a long time, the driver shifts the line of sight for a long time, the driver screams, the driver's face is fearsome, etc., which can all be used as the judgment criteria for the abnormal driving information. If the body state information of the driver is the abnormal driving information, the vehicle may be controlled to execute the corresponding security action, such as: deceleration, braking, alarming, opening the gasbag or the like, so as to reduce or avoid injury to the driver.

Embodiments of the present disclosure provide the vehicle control method for driving security of the driver. In conventional technologies, the driver often loses control of the vehicle often due to subjective reasons of the driver in driving, causing a serious vehicle accident and seriously affecting the security of the society. Compared with the conventional technologies, the vehicle control method provided by the present application includes: first, acquiring the body state information of the driver, and then judging whether the body state information is the abnormal driving information, and controlling the vehicle to execute the corresponding security action if the body state information is the abnormal driving information, so that in a situation where the driver experiences a subjective runaway phenomenon during driving, such as falling asleep, screaming or the like, the vehicle control system automatically controls the vehicle to execute the security action, to ensure the security of the driver.

For example, as shown in FIG. 7, the vehicle control method further includes: step 121, acquiring the vehicle state information of the vehicle; and step 122, judging whether the vehicle state information is the abnormal vehicle information, and judging whether the body state information is the abnormal driving information, in which step, if the vehicle state information is the abnormal vehicle information, and/or the body state information is the abnormal driving information, the vehicle is controlled to execute the corresponding security action.

It can be seen that, in addition to judging based on the body state information of the human body, it is also possible to judge a state of the vehicle based on the vehicle state information; even if the body state information of the human body is normal, the vehicle is also controlled to execute the corresponding security action if the vehicle state information is the abnormal vehicle information, which is beneficial to further improving the security of driving the vehicle.

For example, the vehicle state information may also be various, such as collision information of the vehicle, vibration information of the vehicle, tilt angle information of the vehicle, water pressure information of the outer part of the vehicle and the like, which may all be used as the vehicle state information. The abnormal vehicle information involves a situation that the vehicle state information is abnormal, and the situation is that, for example: collision occurs to the vehicle, the vibration amplitude of the vehicle is large, the tilt angle of the vehicle is large, or the water pressure at the outer part of the vehicle is large; in this situation, the vehicle may be controlled to execute the corresponding security action, and the detailed of the actions are described in detail in the above embodiments and are not described here.

Further, the body state information includes at least one selected from the group consisting of sound information, facial expression information, eyeball information, the heart rate information and brain wave information; the vehicle state information includes at least one selected from the group consisting of collision information, vibration information, tilt information and water pressure information; the security action includes at least one selected from the group consisting of braking action, deceleration action, the gasbag opening action and glass breaking action. In the embodiment, the body state information, the vehicle state information and the security action are described in detail in the foregoing device embodiments, and the working principle and the process are same as those in the device embodiment and are not described here.

The vehicle control method provided by the embodiments of the present disclosure may be implemented by, for example, a combination of hardware and software, which is not limited by the embodiments of the present disclosure.

What are described above is related to the illustrative embodiments of the disclosure only and not limitative to the scope of the disclosure; the scopes of the disclosure are defined by the accompanying claims.

1. A vehicle control system, comprising:
an acquiring device configured to acquire body state information of a driver of a vehicle;
a processor, wherein an input terminal of the processor is connected with the acquiring device, and the processor is configured to judge whether the body state information is body abnormality information, and to output an execution signal in a case where the body state information is the body abnormality information; and
an execution device, wherein an input terminal of the execution device is connected with an output terminal of the processor to receive the execution signal and to execute a corresponding security action according to the execution signal.

2. The vehicle control system according to claim 1, further comprising:
a vehicle detector configured to detect vehicle state information of the vehicle;
wherein the input terminal of the processor is further connected with the vehicle detector, and the processor is further configured to judge whether the vehicle state information is vehicle abnormality information, and to output the execution signal in a case where the vehicle state information is the vehicle abnormality information.

3. The vehicle control system according to claim 1, wherein
the acquiring device comprises a detection device which is configured to be in the vehicle and to be opposite to a front of a position of the driver.

4. The vehicle control system according to claim 3, wherein the detection device comprises at least one selected from a group consisting of a sound detector, a face collecting device and an eyeball detector, and the body state information correspondingly comprises at least one selected from a group consisting of sound information, facial image information and eyeball information;
the sound detector is configured to detect the sound information in the vehicle, the face acquiring device is configured to acquire the facial image information, and the eyeball detector is configured to acquire the eyeball information.

5. The vehicle control system according to claim 4, wherein
the sound detector comprises a microphone, the eyeball detector comprises an infrared light emitter and an eyeball position sensor, and the face acquiring device comprises a camera.

6. The vehicle control system according to claim 1, wherein
the acquiring device further comprises a brain wave detector which is configured to be worn on a head of the driver, and an output terminal of the brain wave detector is connected with the processor;
the brain wave detector is configured to detect brain wave information of the driver, which is used as the body state information.

7. The vehicle control system according to claim 2, wherein the vehicle detector comprises at least one selected from a group consisting of a vibration detector, a collision detector, a tilt detector and a water pressure detector;
the collision detector is configured to detect collision information of the vehicle, which is used as the vehicle state information;
the vibration detector is configured to detect vibration information of the vehicle, which is used as the vehicle state information;
the tilt detector is configured to detect tilt information of the vehicle, which is used as the vehicle state information; and
the water pressure detector is configured to detect vehicle body water pressure information of the vehicle, which is used as the vehicle state information.

8. The vehicle control system according to claim 1, wherein
the execution device comprises at least one selected from a group consisting of a brake device, a gasbag opener, a glass breaker and an alarm.

9. A vehicle, comprising the vehicle control system according to claim 1.

10. A vehicle control method, comprising:
acquiring body state information of a driver of a vehicle;
judging whether the body state information is abnormal driving information, and controlling the vehicle to execute a corresponding security action in a case where the body state information is the abnormal driving information.

11. The vehicle control method according to claim 10, wherein judging whether the body state information is the abnormal driving information, and controlling the vehicle to execute the corresponding security action in case where the body state information is the abnormal driving information, comprises:
acquiring vehicle state information of the vehicle;
judging whether the vehicle state information is abnormal vehicle information;
judging whether the body state information is the abnormal driving information; and
controlling the vehicle to execute the corresponding security action in a case where the vehicle state information is the abnormal vehicle information, and/or, the body state information is the abnormal driving information.

12. The vehicle control method according to claim 10, wherein
the body state information comprises at least one selected from a group consisting of sound information, facial expression information, eyeball information, heart rate information and brain wave information;
the security action comprises at least one selected from a group consisting of a braking action, a deceleration action, a gasbag opening action and a glass breaking action.

13. The vehicle control method according to claim 10, wherein
the vehicle state information comprises at least one selected from a group consisting of collision information, vibration information, tilt information and water pressure information.

14. The vehicle control system according to claim 2, wherein
the acquiring device comprises a detection device which is configured to be in the vehicle and to be corresponding to a position of the driver.

15. The vehicle control system according to claim 14, wherein the detection device comprises at least one selected from a group consisting of a sound detector, a face collecting device and an eyeball detector, and the body state information correspondingly comprises at least one selected from a group consisting of sound information, facial image information and eyeball information;
the sound detector is configured to detect the sound information in the vehicle, the face acquiring device is configured to acquire the facial image information, and the eyeball detector is configured to acquire the eyeball information.

16. The vehicle control system according to claim 2, wherein
the acquiring device further comprises a brain wave detector which is configured to be worn on a head of the driver, and an output terminal of the brain wave detector is connected with the processor;
the brain wave detector is configured to detect brain wave information of the driver, which is used as the body state information.

17. The vehicle control system according to claim 3, wherein
the acquiring device further comprises a brain wave detector which is configured to be worn on a head of the driver, and an output terminal of the brain wave detector is connected with the processor;
the brain wave detector is configured to detect brain wave information of the driver, which is used as the body state information.

18. The vehicle control system according to claim 4, wherein
the acquiring device further comprises a brain wave detector which is configured to be worn on a head of the driver, and an output terminal of the brain wave detector is connected with the processor;
the brain wave detector is configured to detect brain wave information of the driver, which is used as the body state information.

19. The vehicle control system according to claim 5, wherein
the acquiring device further comprises a brain wave detector which is configured to be worn on a head of the driver, and an output terminal of the brain wave detector is connected with the processor;
the brain wave detector is configured to detect brain wave information of the driver, which is used as the body state information.

20. The vehicle control method according to claim 11, wherein
the body state information comprises at least one selected from a group consisting of sound information, facial expression information, eyeball information, heart rate information and brain wave information;
the security action comprises at least one selected from a group consisting of a braking action, a deceleration action, a gasbag opening action and a glass breaking action.
